(19) United States
(12) Patent Application Publication
(10) Pub. No.: US 2008/0310145 A1
(43) Pub. Date: Dec. 18, 2008

(54) Personal Lighting Apparatus

(75) Inventors: John Thirlwall Blake, Kenmore (AU); Neil Davidson, Toowong (AU)

Correspondence Address: WORKMAN NYDEGGER, 60 EAST SOUTH TEMPLE, 1000 EAGLE GATE TOWER, SALT LAKE CITY, UT, 84111, US

(73) Assignee: JOHN BLAKE PRACTICE MANAGEMENT PTY. LTD., COOPERS PLAINS (AU)

(21) Appl. No.: 11/909,438

(22) PCT Filed: Mar. 23, 2006
(86) PCT No.: PCT/AU2006/000388

§ 371 Date: Apr. 18, 2008

(30) Foreign Application Priority Data
Mar. 23, 2005 (AU) 2005901428

(51) Int. Cl. F21V 21/084; A61B 1/06

(52) U.S. Cl. 362/105

(57) ABSTRACT

A personal lighting apparatus (100) is disclosed. The personal lighting apparatus has a light source (131) and a non-contact switching device (123) in operative communication with the light source in order to actuate the light source between an on position and an off position. Additionally, the personal lighting apparatus has a mounting apparatus (110) adapted to locate the personal light apparatus on a user such that light emitted by the light source is directed substantially in a direction of the user's field of view.

### FIELD OF THE INVENTION

The invention relates to a personal lighting apparatus. In particular, although not exclusively, the invention relates to a personal lighting apparatus for use in medical and dental surgical applications.

### BACKGROUND TO THE INVENTION

The use of constant lighting devices during medical and dental procedures is essential in order that medical professionals may have an unshaded view of the area upon which they are performing surgery.

Additionally, in dental surgical applications there exists the need to focus a relatively narrow beam of light within a person's mouth in order that the dental professional is able to clearly see the surgical procedure which they are performing without shadowing effects causing a disturbance.

Furthermore, there is a need for dental professionals to be able to turn their surgical light on and off without having to contact the light. This is due to the fact that the dental professionals sterilise before a procedure and risk contamination of their sterilised hands if physical contact with a light switch is required whilst performing a procedure.

U.S. Pat. No. 5,637,863 discloses a light fixture for use in a sterile environment wherein the housing has a touchless switch means operatively connected to toggle the light fixture in order that a medical professional is able to turn the light fixture on and off without having to physically contact the switch and hence risk contamination.

The invention described in U.S. Pat. No. 5,637,863 is for particular use in medical surgical applications and hence is designed for a fixed location lighting apparatus as is known in the art. However, this invention is deficient when applied to dental procedures as it does not allow for a directed beam of light into a persons mouth.

For example, if it is required to change the orientation of a person's head during a dental procedure, it is necessary to refocus the light in order that a narrow beam of light is directed within the person's mouth. In order to achieve this change in direction when using the U.S. Pat. No. 5,657,863 invention it is necessary to physically move the orientation of the light fixture and hence the dental professional risks contamination of their hands.

U.S. Pat. No. 4,538,070 discloses an apparatus for treating dental prosthetic parts with radiation. The apparatus includes a lamp, a reflector, a filter and an optical wave guide to project radiation of a selected spectral range onto a location of treatment. When an object is placed at this location of treatment, the object actuates an infrared proximity switch which turns on the lamp for at least a predetermined period of time.

This apparatus is directed to treatment of dental prosthetic parts and is mounted upon a fixed surface. As such, the U.S. Pat. No. 4,538,070 device does not address the shadowing effect that occurs during dental procedures.

U.S. Pat. No. 4,195,918 relates to illuminating spectacles with variable magnifying power comprising a spectacle frame provided with removable illumination means. The removable illumination means consists of light conduits resiliently engaged in grooves provided in the top of the frame and on inner edges of the frame on either side of the nose. Each light conduit terminates at a lens such that light is directed in the axis of vision of the working field.

The illuminating spectacles disclosed in U.S. Pat. No. 4,195,918 are cumbersome in that the light conduits are connected to a source of light mounted on a fixed structure and hence the light conduits can cause interference to the user if, for example, they turn their head causing the light conduits to pull on the frame.

Furthermore, it is necessary to use physical contact to actuate the light switch and thus there exists the potential for contamination of the dental practitioners hand as discussed above. As such, the device disclosed in U.S. Pat. No. 4,195,918 is not suited for use in dental procedures.

Hence, it is desirable to provide a lighting apparatus for dental procedures that does not risk contamination of the dental professionals hand when switching the light on and off and is able to be easily directed within a patients mouth regardless of whether the orientation of the patient's head changes during the procedure.

### OBJECT OF THE INVENTION

It is an object of the invention to overcome or at least alleviate one or more of the above problems or provide the consumer with a useful or commercial choice.

### DISCLOSURE OF THE INVENTION

In one form, although it need not be the only or indeed the broadest form, the invention resides in a personal lighting apparatus comprising:

a light source;

a non-contact switching device in operative communication with said light source in order to actuate said light source between an on position and an off position; and

a mounting apparatus adapted to locate said personal lighting apparatus on a user such that light emitted by said light source is directed substantially in a direction of said user's field of focus.

Further features of the present invention will become apparent from the following detailed description.

### BRIEF DESCRIPTION OF THE DRAWINGS

To assist in understanding the invention and to enable a person skilled in the art to put the invention into practical effect preferred embodiments of the invention will be described by way of example only with reference to the accompanying drawings, wherein:

FIG. 1 shows a schematic side view of a personal lighting apparatus according to an embodiment of the invention;

FIG. 2 shows a base station for use with the personal lighting apparatus shown in FIG. 1;

FIG. 3A shows a front perspective view of an embodiment of the personal lighting apparatus of the invention;

FIG. 3B shows a rear perspective view of the personal lighting apparatus shown in FIG. 3A;

FIG. 4A shows a front perspective view of the personal lighting apparatus shown in FIG. 3A fitted to a head of a user;

FIG. 4B shows a rear perspective view of the personal lighting apparatus shown in FIG. 3A fitted to a head of a user;

FIG. 5A shows a front perspective view of a further embodiment of the personal lighting apparatus of the invention;

FIG. 5B shows a rear perspective view of the personal lighting apparatus shown in FIG. 5A;

FIG. 6A shows a front perspective view of the personal lighting apparatus shown in FIG. 5A fitted to a head of a user;

FIG. 6B shows a rear perspective view of the personal lighting apparatus shown in FIG. 5A fitted to a head of a user;

FIG. 7A shows a front perspective view of a further embodiment of the personal lighting apparatus of the invention;

FIG. 7B shows a rear perspective view of the personal lighting apparatus shown in FIG. 7A;

FIG. 8A shows a front perspective view of the personal lighting apparatus shown in FIG. 7A fitted to a head of a user;

FIG. 8B shows a rear perspective view of the personal lighting apparatus shown in FIG. 7A fitted to a head of a user;

FIG. 9A shows a front perspective view of a further embodiment of the personal lighting apparatus of the invention fitted to a head of a user; and

FIG. 9B shows a rear perspective view of the personal lighting apparatus shown in FIG. 9A.

### DETAILED DESCRIPTION OF THE INVENTION

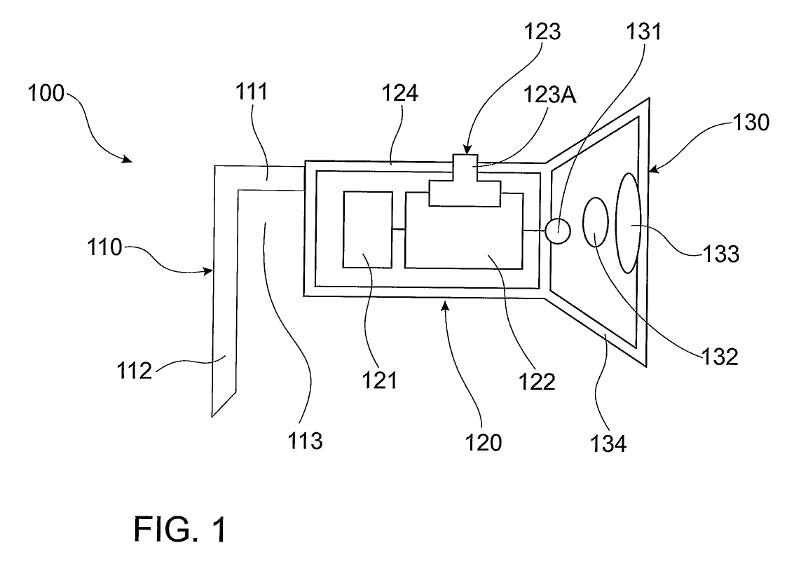

FIG. 1 shows a schematic side view of a personal lighting apparatus 100 according to an embodiment of the invention. The personal lighting apparatus comprises a body 120, a mounting apparatus 110 located on a side of body 120 and a lighting unit 130 located on a side of body 120.

Body 120 comprises a hollow housing 124 formed from a resilient, shock resistant material. Preferably, hollow housing 124 is formed from plastic, fiberglass, alloys of plastic, fiberglass, metal alloys or the like. Preferably, hollow housing 124 is adapted to facilitate access to an internal area of body 120. Optionally, hollow housing is water sealed to minimize the possibility of water seepages within an internal area of body 120. Alternatively, hollow housing 124 may be formed from a single integral member.

Body 120 further comprises a processing module 122 located within the internal volume of body 120 formed by hollow housing 124. Preferably, processing module 122 is in the form of a printed circuit board (PCB) having input/output circuitry, power distribution circuitry and control circuitry. Optionally, processing module 122 may be any form of logic control device known in the art.

Switching device 123 protrudes through body 120 and is located within the internal area of body 120 formed by hollow housing 124. Switching device 123 is in electrical communication with processing module 122. Switching device 123 has a detection unit 123A extending to an outside of body 120.

Preferably, switching device 123 is in the form of an infrared detection device whereby switching device 123 is adapted to receive a non-contact user command by observing a gesture in the proximity of the detection unit 123A. Alternatively, switching device 123 may be in the form of magnetic sensors such as Hall Effect sensors or the like.

Body 120 further comprises a power module 121. Power module 121 is in electrical communication with processing module 122 and provides power to the electrical circuitry of processing module 122, switching device 123 and lighting unit 130.

Preferably, power module 121 is formed from one or more rechargeable batteries in electrical contact with a power distribution circuit located within the processing module 122. Power module 121 has shielded electrical contacts (not shown) protruding outside of hollow housing 124 in order that the rechargeable batteries that form part of power module 121 may be recharged.

Optionally, power module 121 may have replaceable batteries located on battery contacts as is known in the art. In this embodiment of personal lighting apparatus 100, the replaceable batteries are removed by disassembling housing 124 as previously described. Alternatively, these batteries may be in the form of cartridge type batteries that may be removed from personal lighting apparatus 100 for recharging without the need to disassemble housing 124.

It will be appreciated that power module 121 may be physically separate from body 120 such that power is provided to body 120 and lighting unit 130 by means of power cables or the like.

Mounting apparatus 110 is securely attached to a side of hollow housing 124 of body 120. Optionally, mounting apparatus 110 is integrally formed with hollow housing 124 of body 120. Preferably, mounting apparatus 110 is formed from a durable, rigid plastic. Alternatively, mounting apparatus 110 may be formed from a flexible material.

Mounting apparatus 110 is formed from an extending member 111 and a retaining member 112 integrally formed with extending member 111 and hence a cavity 113 is formed by extending member 111, retaining member 112 and a side of hollow housing 124 of body 120. The function of mounting apparatus 110 will be discussed below.

Lighting unit 130 comprises a housing 134 securely attached to hollow housing 124 of body 120. Optionally, housing 134 may be integrally formed with hollow housing 124 of body 120.

Alternatively, housing 134 of lighting unit 130 may be pivotally mounted upon body 120 as will be discussed in greater detail below.

A light source 131 forms part of lighting unit 130. Light source 131 is electrically connected to processing module 122 of body 120. Hence, processing module 122 operatively provides power to light source 131 via power module 121 and also controls the operation of light source 131.

Light source 131 is able to be actuated between an on position where it emits a beam of light, and an off position where it does not emit a beam of light. Light source 131 is actuated in response to a non-contact user command by observing a gesture in the proximity of the detection unit 123A.

Suitably, the detection unit 123A of the switching device is able to be biased to an activation direction and a sensitivity range. For example, a right handed surgeon will have a dental assistant working on their left hand side. In order to avoid false reads caused by the detection unit 123A observing movement by the dental assistant, the detection unit 123A is biased to only detect user commands on a right side of the surgeon. Preferably, the biasing of the activation direction is controlled by moveable mechanical shields or partitions in order that the biasing direction may be selected. Alternatively, the biasing of the activation direction may be facilitated by a moveable lens at the detection unit 123A.

Preferably, the read range of the detection unit 123A is selected such that an effective read range of between 0 and 300 mm from the detection unit 123A is provided.

Preferably, light source 131 is in the form of a light emitting diode (LED) appropriate for dental work. As such, light source 131 emits light at a colour temperature of approximately 5500 degrees Kelvin and a lux value of 8000 or greater at the target. This colour temperature allows a dental practitioner to accurately make standardized shade selections for dental restoratives, such as fillings, crowns, veneers and dentures without turning the light source off.

Lighting unit 130 further comprises a focusing mechanism 132,133 in order that the light emitted from light source 131 may be narrow and selectively directed. Preferably, the focusing mechanism emits a focused disc of light 60 mm in diameter such that the light from the light source enters the cavity in the patient's mouth without projecting on the patient's eye.

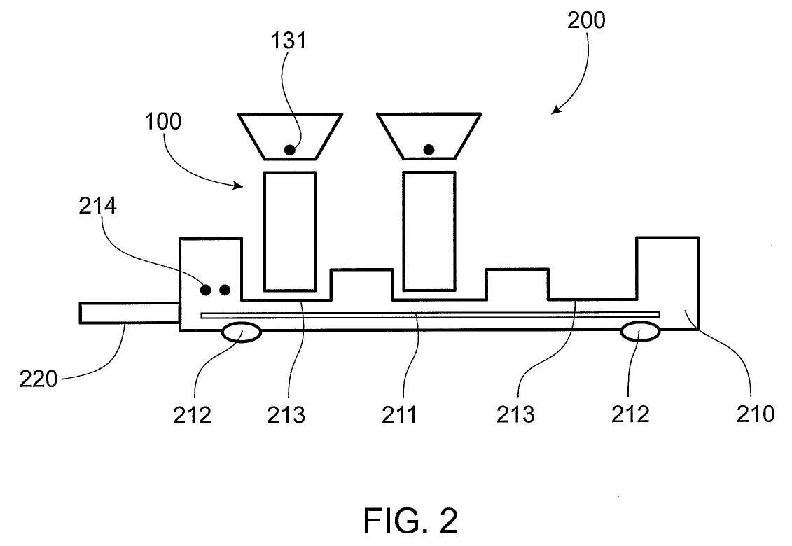

FIG. 2 shows a base station 200 for the personal lighting apparatus 100 shown in FIG. 1. Base station 200 comprises a base 210 and an electrical power input 220 in electrical contact with base 210. Electrical power input 210 is a standard electrical power lead providing power to base station 200.

Base 210 has a control module 211 in electrical contact with electrical power input 220. Base 210 further comprises a plurality of feet 212, a series of cavities 213 and one or more indication lights 214. Each cavity 213 is adapted to receive a personal lighting apparatus 100 and has located on a side an electrical contact (not shown) mateable with the electrical contact (not shown) on each personal lighting apparatus previously described.

In use, when a personal lighting apparatus 100 is located within a cavity 213 and the respective electrical contacts are mated, control module 211 routes the power supply received from electrical power input 220 to the personal lighting apparatus 100 in order to recharge the power module 121. Additionally, control module 211 indicates that the personal lighting apparatus 100 is being recharged via indication lights 214.

During recharge, the control module 211 monitors the strength of the power module 121 of the personal lighting apparatus 100. When the power module 121 has reached full capacity, control module 121 blocks any further power source from reaching power module 121 and indicates, via indication lights 211, that the personal lighting apparatus 100 has been recharged. It will be appreciated that there will be indication lights 214 for each cavity 213 on base 210.

Lighting apparatus 100 is used by medical practitioners and in particular dental practitioners during dental procedures. Mounting apparatus 110 is adapted to locate the personal lighting apparatus 100 on a medical or dental practitioner. The practitioner places the retaining member 112 between the bridge of their safety or prescription glasses and the bridge of their nose until extending member 111 is located on the top of their glasses bridge. As such, cavity 113 receives the bridge of the practitioner's glasses and the location of extending member 111 along the bridge of the practitioner's nose firmly holds the personal lighting apparatus on the practitioner's face such that the light is directed towards the centre of the practitioner's field of focus and the light source is located between, and in line with, the practitioner's eyes. Hence, the possibility of shading caused by a light source emitted from a different plane is reduced as the light source is directed within a patient's mouth without the possibility of shadowing occurring.

It will be appreciated that mounting apparatus 110 may be in other forms than that described herein. For example, mounting apparatus may take the form of a head band adapted to hold the personal lighting apparatus 100 on a practitioners head or any other form of mounting means to mount the personal lighting apparatus 100 to a user's head.

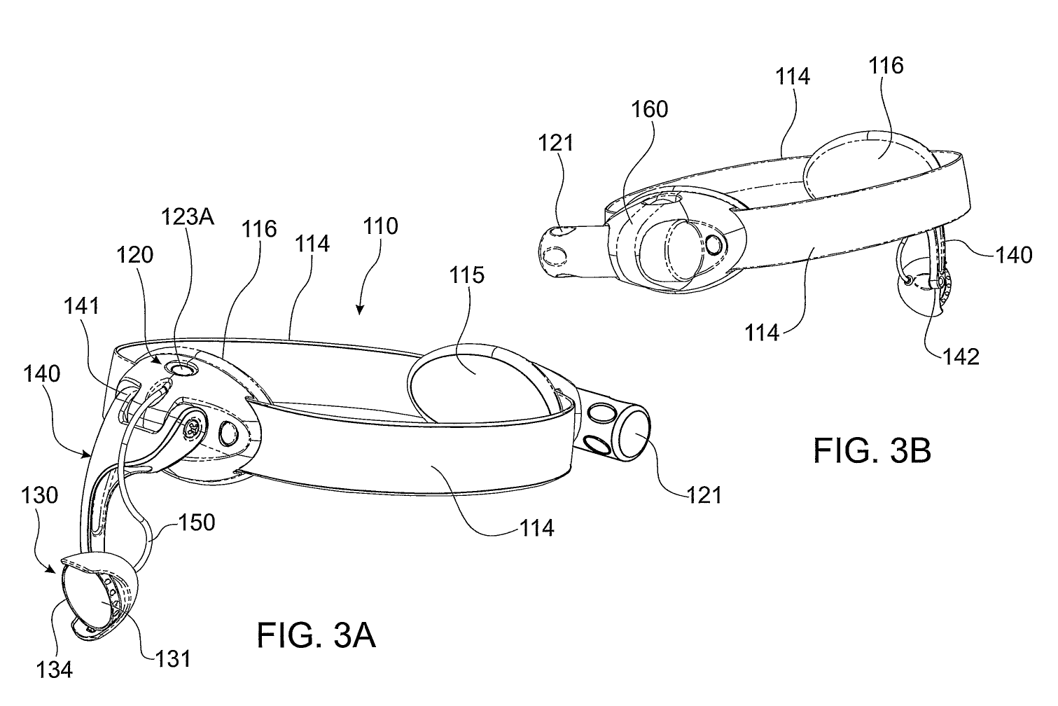

FIG. 3A shows a front perspective view of an embodiment of the personal lighting apparatus 100 of the invention and FIG. 3B shows a rear perspective view of the personal lighting apparatus 100 shown in FIG. 3A.

In this embodiment, the mounting apparatus 110 is in the form of a head band 114 having a rear support pad 115 located upon a rearward region of head band 114 and forward support pad 116 located upon a forward region of head band 114. Body 120 is located upon an outer face of forward support pad 116. Suitably, the length of head band 114 is adjustable in order that the personal lighting apparatus 100 is securely mounted upon the head of a user.

Additionally, a power module mount 160 is located upon an outer face of support pad 115. Power module 121 in the form of a rechargeable battery which is removably mounted on power module mount 160. The power module 121 is in electrical communication with body 120 to provide electrical power. Preferably, this electrical communication is in the form of electrical cables woven into head band 114. Optionally, an electrical cable is securely attached in order that power is transited from power module 121 to body 120 and to lighting unit 130.

Detection unit 123A of switching device 123 is located upon body 120 as shown. Furthermore, an adjustment arm 140 is pivotally mounted upon body 120 by means of a pivot 141. Lighting unit 130 is pivotally mounted upon adjustment arm 140 by means of a pivot 142.

A data and power cable 150 extends from body 120 to lighting unit 130 in order that power is transmitted to light source 131 of lighting unit 130 and light source 131 can be actuated in response to non-contact actuation commands received at detection unit 123A of switching device 123 as previously discussed.

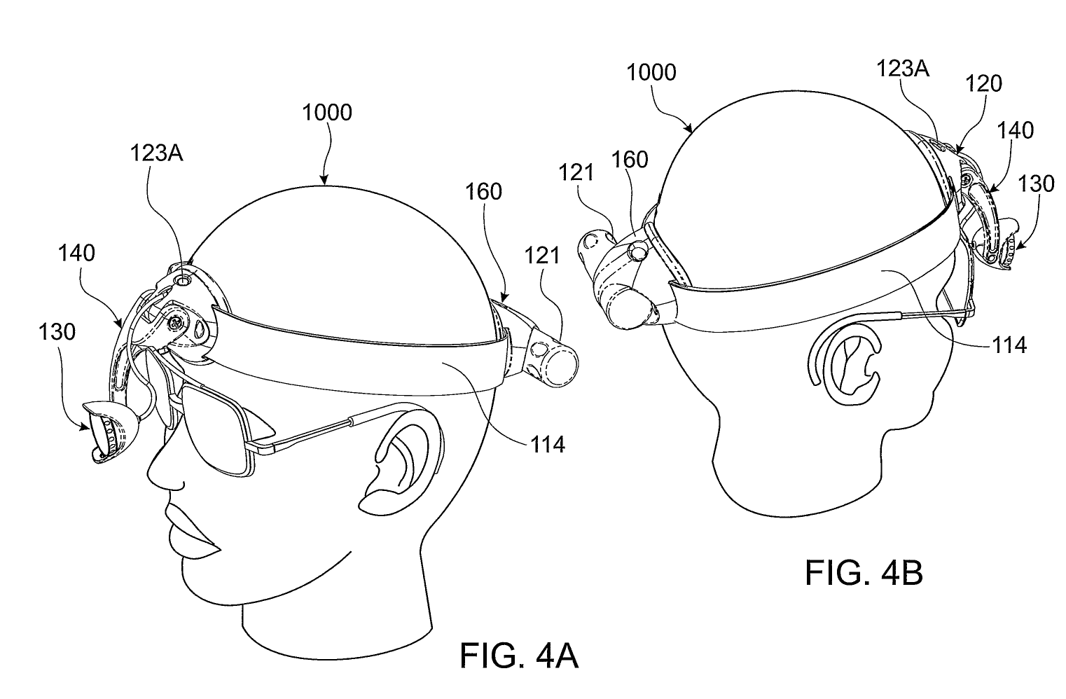

FIG. 4A shows a front perspective view of the personal lighting apparatus 100 shown in FIG. 3A fitted to a head of a user and FIG. 4B shows a rear perspective view of the personal lighting apparatus shown in FIG. 3A fitted to a head of a user.

The forward support pad 116 rests on a forehead of the user 1000 and the rear support pad 115 rests upon a rear of the head of the user 1000. The presence of the power module mount 160 and the power module 121 in the form of a rechargeable battery acts as a counter weight for the body 120, lighting unit 130 and location arm 140 in order that the personal lighting apparatus 100 sits securely on the head of the user 1000.

As shown, the light from the light source 131 is directed substantially in a direction of the user's field of focus and the vertical alignment of the lighting unit 131 is moveable by rotating the location arm 140 with respect to the body 130 about pivot 141. Furthermore, the vertical direction of the light emitted from the light source 131 is able to be adjusted by rotating the lighting module 130 with respect to location arm 140 about pivot 142.

In this way, user 1000 is able to adjust the direction of the light emitted by the light source 131 such that it is directed substantially in a direction of the user's field of focus.

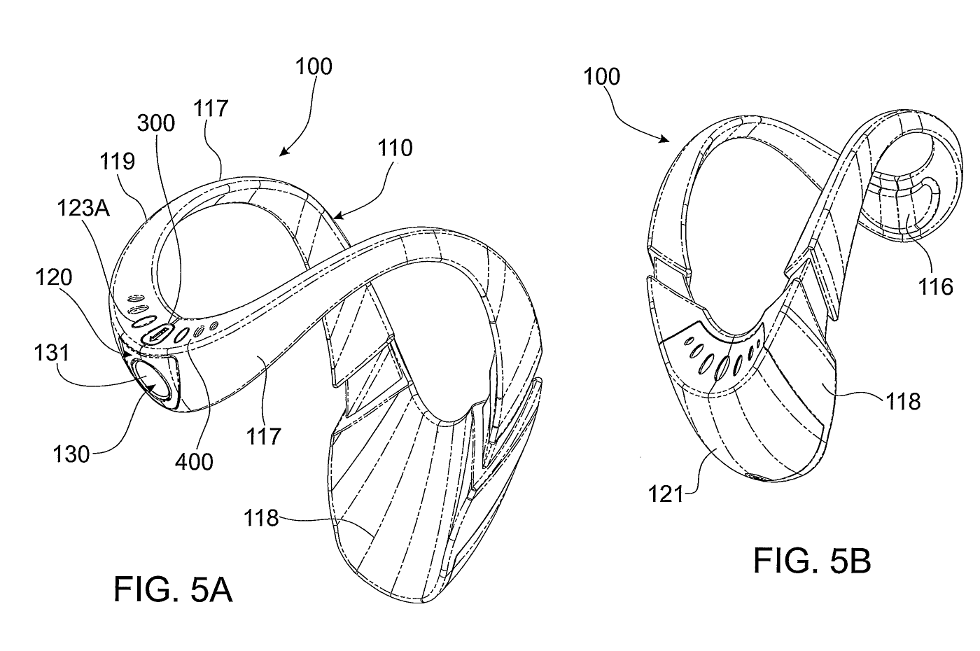

FIG. 5A shows a front perspective view of a further embodiment of the personal lighting apparatus 100 of the invention and FIG. 5B shows a rear perspective view of the personal lighting apparatus shown in FIG. 5A.

In this embodiment, the mounting apparatus 110 is formed from a rigid durable material such as a hard plastic or the like. The mounting apparatus has a pair of curved side rails 117 extending rearwardly from a forehead section 119. Curved side rails 117 terminate at a rear of the person's head at a cradle 118. Cradle 118 has an inner face for location on a base of the head of a user.

Body 120 and lighting unit 130 are located within forehead section 119 as shown. Additionally, a forehead location pad 116 is located on an inner face of forehead section 116.

Power module 118 is located on a rear face of cradle 118 and is in electrical communication with body 120 to provide electrical power thereto. Preferably, electrical cables extend through an inner cavity of one or both of curved side rails 117 to body 120 and lighting unit 130.

Detection unit 123A of switching unit 123 is located on an upper surface of forehead section 119 and an adjustment button 300 is located on an upper surface of forehead section 119. Adjustment button 300 is able to control the vertical and horizontal direction of light emitted from light source 131 in order that a user is able to direct the light source such that the light emitted is directed substantially in a direction of the user's field of focus.

Additionally, a plurality of indicator lights 400 are located upon mounting apparatus 110 in order to provide an indication to the user of such information as the battery status and the like.

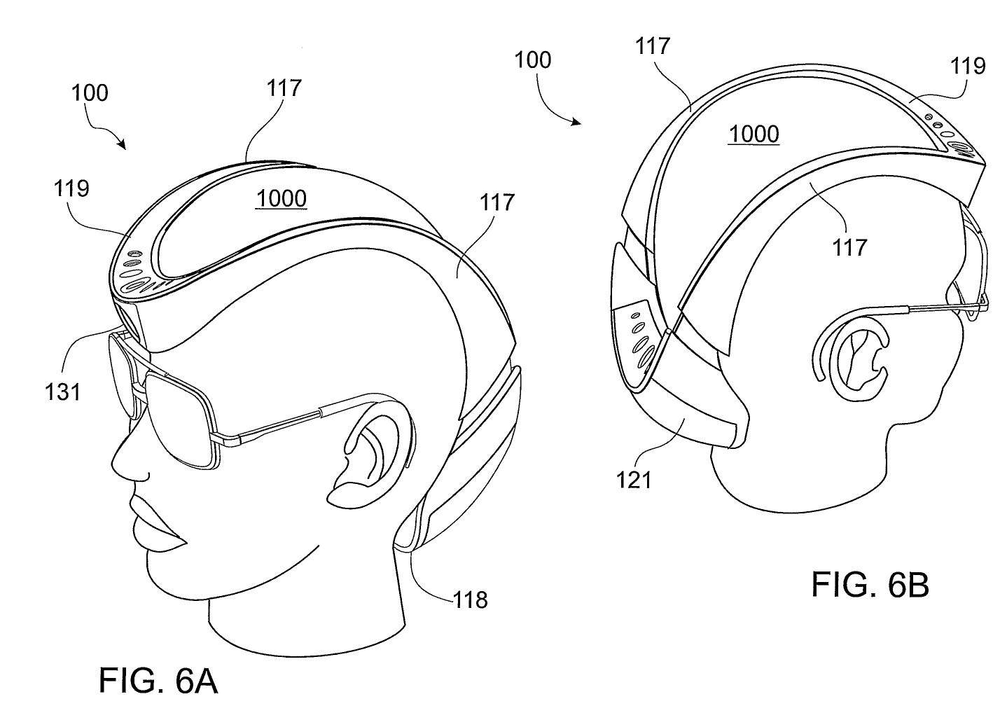

FIG. 6A shows a front perspective view of the personal lighting apparatus 100 shown in FIG. 5A fitted to a head of a user 1000 and FIG. 6B shows a rear perspective view of the personal lighting apparatus 100 shown in FIG. 5A fitted to a head of a user 1000.

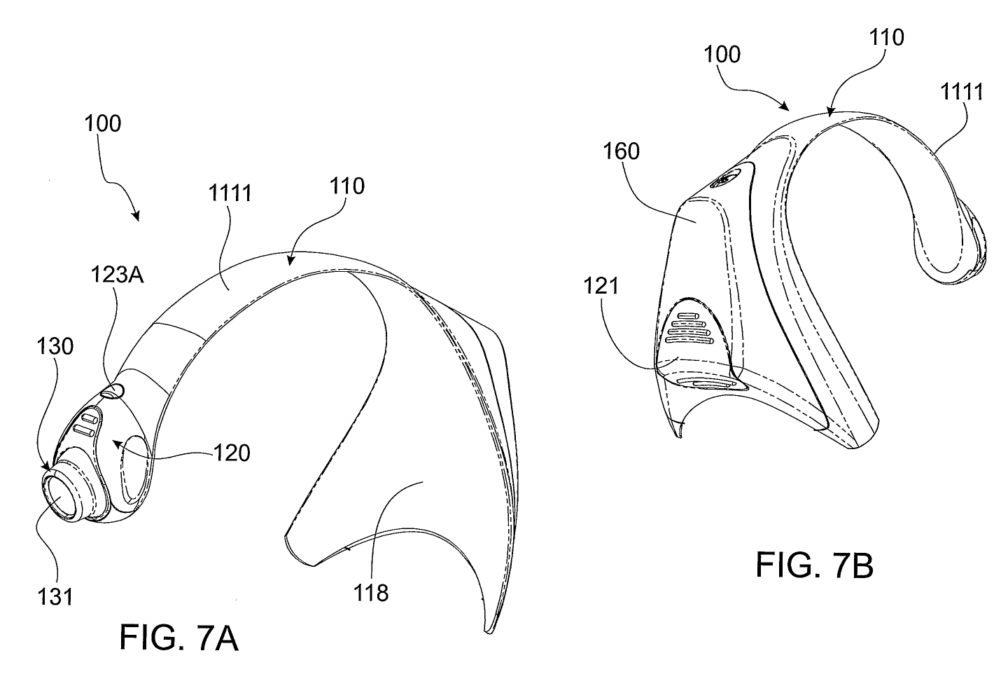

FIG. 7A shows a front perspective view of a further embodiment of the personal lighting apparatus 100 of the invention and FIG. 7B shows a rear perspective view of the personal lighting apparatus 100 shown in FIG. 7A. Mounting apparatus is in the form of a central mounting rail 1111 formed from a rigid durable material such as plastic or the like.

Central mounting rail extends from a front region of a head of a user to rear region and has body 120 located at a front end and a cradle 118 located at a rear end thereof.

Lighting unit 130 is rotatably mounted upon body 120 in order that the direction of light emitted from light source 131 may be changed with respect to a vertical axis. Optionally, lighting unit 130 may also be horizontally mounted upon body 120 in order that the direction of light emitted from light source 131 may be changed with respect to a horizontal axis. As shown, detection unit 123A of switching device 123 is located on an upper side of body 120.

A power module mount 160 is located upon an outer face of cradle 118 and power module 121 in the form of a rechargeable battery is removably mated therewith. Power module 121 and power module mount 160 is in electrical communication with body 120 and lighting unit 130 in order that electrical power is supplied to body 120 and thereby to lighting unit 130.

Preferably, this electrical communication is facilitated by way of electrical cables extending within central mounting rail 1111 from power module 121 and power module mount 160 to body 120.

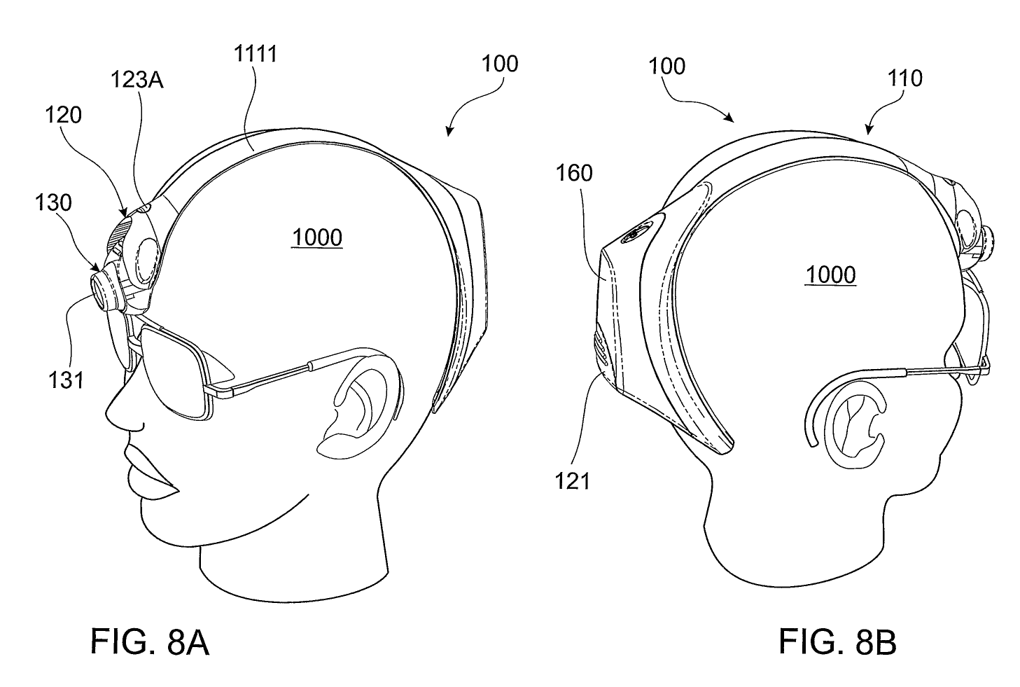

FIG. 8A shows a front perspective view of the personal lighting apparatus 100 shown in FIG. 7A fitted to a head of a user 1000 and FIG. 8B shows a rear perspective view of the personal lighting apparatus 100 shown in FIG. 7A fitted to a head of a user 1000. As shown in FIG. 8A, the lighting unit 130 has been rotated downwardly with respect to body 120 such that the light source 131 is directed within the user's field of focus.

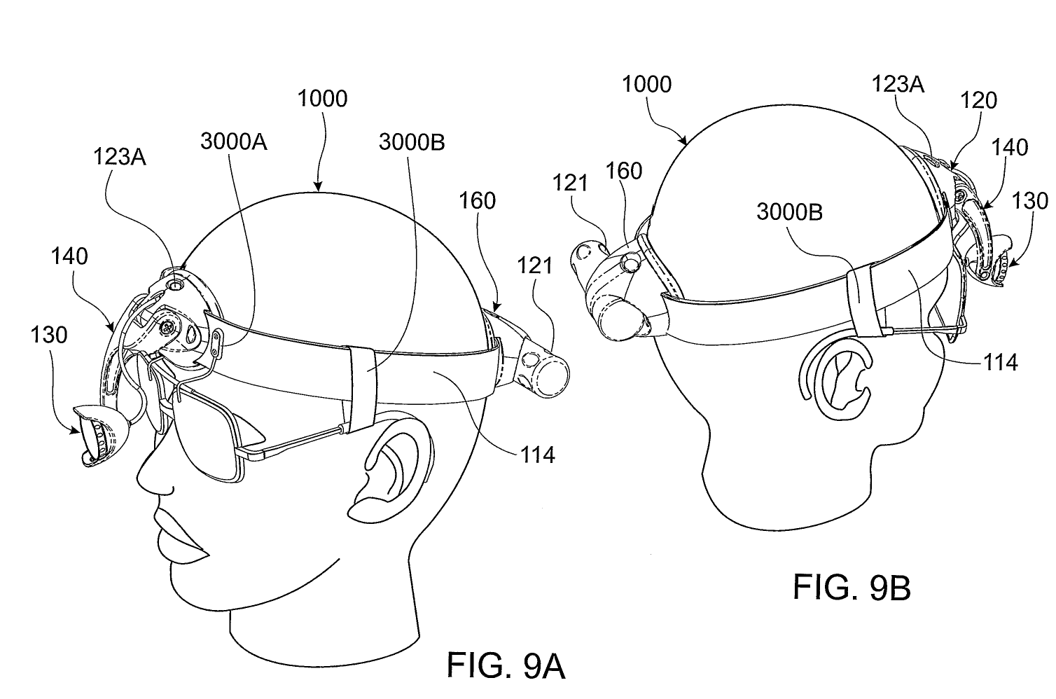

FIG. 9A shows a front perspective view of a further embodiment of the personal lighting apparatus 100 of the invention fitted to a head of a user and FIG. 9B shows a rear perspective view of the personal lighting apparatus 100 shown in FIG. 9A.

The embodiment shown in FIGS. 9A and 9B is the same as that shown in FIGS. 3A to 4B with the inclusion of rigid adaptors 3000A and non-rigid adaptors 3000B secured to mounting apparatus 110. The non-rigid adaptors 3000B are used to secure, for example, the user's 1000 prescription glasses onto their head. Furthermore, the rigid adaptors 3000A are used to further locate the prescription glasses of the user 1000 on their head.

It will be appreciated that features of the embodiments of the invention are exemplary and are not intended to be limiting. Certain features of one embodiment may be included in another embodiment without departing from the spirit and scope of the invention.

Personal lighting apparatus 100 provides for a lighting apparatus for use in dental procedures that does not risk contamination of the dental professional's hand when switching the light between on and off. Furthermore, due to the location of the personal lighting apparatus 100 of the present invention on the head of a practitioner, the beam of light emitted by light source 131 can easily be directed within a patient's mouth as it always shines in the centre of the practitioner's field of focus.

Additionally, as the personal lighting apparatus 100 of the present invention has contained therein its own power source, the possibility of a power lead obstructing the practitioners movements during a procedure is removed.

Throughout the specification the aim has been to describe the invention without limiting the invention to any one embodiment or specific collection of features. Persons skilled in the relevant art may realize variations from the specific embodiments that will nonetheless fall within the scope of the invention. For example, the personal lighting apparatus of the present invention may further include a laser pointer adapted to indicate where the beam of light emitted by light source 131 will be directed when the light source 131 is in the off position.

Furthermore, the personal lighting apparatus of the present invention may further provide pointing capacity by adaptation of the focusing system to produce a dark spot within the beam of light emitted when the light source 131 is in the on position of interest.

Furthermore, it will be appreciated that light source 131 may comprise a plurality of different characteristic light sources with the practitioner being able to selectively emit light from each light source without having to contact personal lighting apparatus 100. For example, light source 131 may have a yellow light for avoiding curing material used in dental operations, such as fillings. Hence, the practitioner is able to selectively swap light sources between a yellow light for specific procedural vision, to a white curing light (colour temperature of approximately 5500-6400 K) for general procedural vision. Suitably, this is facilitated by the detection unit 123A of the switching device 123 toggling between three conditions in response to an activation signal being detected: white light for general procedural vision, yellow light for avoiding curing and off.

Furthermore, it is possible to have located upon the mounting apparatus 110 fastening areas, similar to the adaptors shown in FIG. 9A and FIG. 9B, in order that a user may securely fasten visual and protective aids such as spectacles, operating loupes, splash shields for aseptic operative and non-operative procedures and tools in order to allow for ease of access to these tools during non aseptic procedures and the like.

Additionally, the processing module 122 of the body 120 is able to monitor the power levels of the power module 121 whilst in use. As the power level in power module 121 approaches a low level, the processing module 121 is able to signal to the user that the power in the power module 121 is approaching this low level. Preferably, the processing module 122 flashes the LED 131 off and on at intervals so the operator can see the signal. Additionally, an audible and/or tactile indication is actuated.

Suitably, the signaling may occur at intervals prior to the power in power module being completely drained commencing from a predetermined lower power level.

Whilst the invention has been described in terms of medical and dental applications, it will be appreciated that the invention may have applications in other fields. For example, it may be used by a mechanic or a jeweler.

It will be appreciated that various other changes and modifications may be made to the embodiment described without departing from the spirit and scope of the invention.

1. A personal lighting apparatus comprising:
a lighting unit having a light source;
a non-contact switching device in operative communication with the light source in order to actuate the light source between an on position and an off position; and
a mounting apparatus adapted to locate the personal lighting apparatus on a user such that light emitted by the light source is directed substantially in a direction of the user's field of focus.

2. The personal lighting apparatus of claim 1, further comprising a power module located upon the mounting apparatus for providing electrical power to the light source and the non-contactable switching device.

3. The personal lighting apparatus of claim 1, wherein the light source is pivotable with respect to the mounting apparatus.

4. The personal lighting apparatus of claim 1, wherein the mounting apparatus is in the form of a head band for location on a head of the user.

5. The personal lighting apparatus of claim 4, further comprising a power module mount located upon a rearward region of the head band, the power module mount adapted to have a power module removably mounted thereon, the power module providing electrical power to the light source and the non-contact switching device.

6. The personal lighting apparatus of claim 4, wherein the non-contact switching device is mounted upon a forward region of the head band.

7. The personal lighting apparatus of claim 4, wherein an adjustment arm is pivotally mounted upon a body located upon a forward region of the head band and the light source is pivotally mounted upon the adjustment arm.

8. The personal lighting apparatus of claim 4, wherein a length of the head band is adjustable.

9. The personal lighting apparatus of claim 4, wherein a forward support pad is located upon a forward region of the head band and a rear support pad is located upon a rear region of the head band.

10. The personal lighting apparatus of claim 1, wherein the mounting apparatus is in the form of a pair of curved side rails extending rearwardly from a forehead section of the mounting apparatus.

11. The personal lighting apparatus of claim 10, wherein the pair of curved side rails extend from a forehead section of the mounting apparatus and terminate at a cradle having an inner face for location of a base of a head of the user.

12. The personal lighting apparatus of claim 10, wherein the lighting unit is located within the forehead section.

13. The personal lighting apparatus of claim 10, wherein the personal lighting apparatus further comprises an adjustment button located upon the mounting apparatus, the adjustment button for controlling the vertical and horizontal direction of light emitted from the light source.

14. The personal lighting apparatus of claim 1, wherein the mounting apparatus is in the form of a central mounting rail.

15. The personal lighting apparatus of claim 14, wherein the central mounting rail has a cradle at a rear end, the cradle having an inner face for location of a base of head of the user.

16. The personal lighting apparatus of claim 14, wherein the light source is located at a forward end of the central mounting rail, the light source being moveable in order that light emitted from the light source can be selectively directed vertically and horizontally.

17. The personal lighting apparatus of claim 1, wherein the lighting unit has a focusing mechanism to focus light emitted from the light source into a narrow beam.

18. The personal lighting apparatus of claim 1, wherein the light source is able to be actuated between an on position and an off position and light emitted from the light source able to be further actuated between white light and yellow light in response to a signal received by the non-contact switching device.

19. The personal lighting apparatus of claim 1, wherein the non-contact switching device has a detection unit to enable the non-contact switching device to actuate the light source, the detection unit adapted to receive a user command by observing a gesture proximal the detection device.

20. The personal lighting apparatus of claim 19, wherein the detection device is an infrared detection device.

21. The personal lighting apparatus of claim 19, wherein the detection device is able to be biased to an activation direction.

22. The personal lighting apparatus of claim 19, wherein the detection device is able to be selectively configured to receive a user command in a predetermined range.

23. The personal lighting apparatus of claim 1 further comprising an indicator for indicating when a power level in a power module of the personal lighting apparatus is approaching a predetermined lower threshold.
